(19) United States
(12) Patent Application Publication
(10) Pub. No.: US 2026/0027074 A1
(43) Pub. Date: Jan. 29, 2026

(54) ANIMAL THERAPEUTIC COMPOSITION AND ASSOCIATED METHODS

(71) Applicant: KODISCOVERY, LLC, Baltimore, MD (US)

(72) Inventor: Young Hee KO, Baltimore, MD (US)

(21) Appl. No.: 18/997,349

(22) PCT Filed: Jul. 21, 2023
(86) PCT No.: PCT/US2023/070784

§ 371 Date: Jan. 21, 2025

Related U.S. Application Data
(60) Provisional application No. 63/391,261, filed on Jul. 21, 2022.

(51) Int. Cl. A61K 31/19; A61K 9/00; A61K 47/12; A61K 47/26; A61P 35/00

(52) U.S. Cl. CPC A61K 31/19; A61K 9/0056; A61K 47/12; A61K 47/26; A61P 35/00

(57) ABSTRACT

An animal treatment product is described and discussed. An animal treatment product can include an animal food product and a cellular energy inhibitor composition combined with the animal food product, wherein the cellular energy inhibitor composition includes 3-bromopyruvic acid (3-BP), a 3-BP salt, or a combination thereof, at least one sugar to stabilize the 3-BP molecule by substantially preventing the 3-BP molecule from hydrolyzing, and a biological buffer present in an amount sufficient to at least partially deacidify and neutralize metabolic by-products of the 3-BP.

### CROSS-REFERENCE TO RELATED APPLICATIONS

This application claims the benefit of U.S. Provisional Patent Application No. 63/391,261, filed on Jul. 21, 2022, which is incorporated herein by reference in its entirety.

### SEQUENCE LISTING

This application contains a sequence listing which is incorporated herein by reference in ST.26 XML format named 2553-042-PCT_Sequence_Listing.xml, created Jul. 21, 2023, and is 14 KB in size. The sequences contained in the sequence listing are found throughout the originally filed application.

### BACKGROUND

Pharmaceutical formulations have a wide variety of physical forms and compositional formulations, depending on the active agent in the formulation, the route of administration, etc. For example, a solid pharmaceutical formulation includes an active agent dispersed in a solid pharmaceutical carrier. Similarly, a liquid pharmaceutical formulation includes an active agent dispersed in a liquid pharmaceutical carrier. Additional additives can vary depending on whether the dosage form is a liquid or solid, for example.

### BRIEF DESCRIPTION OF THE DRAWINGS

FIG. 1 illustrates an animal treatment product including an animal food product mixed with a cellular energy inhibitor in accordance with an example embodiment;

FIG. 2 illustrates an animal treatment product including multiple component layers in accordance with an example embodiment;

FIG. 3 illustrates an animal treatment product including an animal food product mixed with a cellular energy inhibitor in accordance with an example embodiment;

FIG. 4 illustrates an animal treatment product including an animal food product mixed with a cellular energy inhibitor in accordance with an example embodiment;

FIG. 5 illustrates an animal treatment product including an animal food product mixed with a cellular energy inhibitor in accordance with an example embodiment;

FIG. 6A illustrates an animal treatment product including an animal food product and a cellular energy inhibitor in packaging in accordance with an example embodiment;

FIG. 6B illustrates an animal treatment product including multiple ingredients separated by packaging in accordance with an example embodiment;

FIG. 7 illustrates animal treatment data over time in accordance with an example embodiment;

FIG. 8 illustrates animal treatment data over time in accordance with an example embodiment; and

FIG. 9A illustrates an image of animal tumor treatment over time in accordance with an example embodiment.

FIG. 9B illustrates an image of animal tumor treatment over time in accordance with an example embodiment.

FIG. 9C illustrates an image of animal tumor treatment over time in accordance with an example embodiment.

### DESCRIPTION OF EMBODIMENTS

Although the following detailed description contains many specifics for the purpose of illustration, a person of ordinary skill in the art will appreciate that many variations and alterations to the following details can be made and are considered included herein. Accordingly, the following embodiments are set forth without any loss of generality to, and without imposing limitations upon, any claims set forth. It is also to be understood that the terminology used herein is for describing particular embodiments only and is not intended to be limiting. Unless defined otherwise, all technical and scientific terms used herein have the same meaning as commonly understood by one of ordinary skill in the art to which this disclosure belongs. Also, the same reference numerals in appearing in different drawings represent the same element. Numbers provided in flow charts and processes are provided for clarity in illustrating steps and operations and do not necessarily indicate a particular order or sequence.

Furthermore, the described features, structures, or characteristics can be combined in any suitable manner in one or more embodiments. In the following description, numerous specific details are provided, such as examples of layouts, distances, network examples, etc., to provide a thorough understanding of various embodiments. One skilled in the relevant art will recognize, however, that such detailed embodiments do not limit the overall concepts articulated herein but are merely representative thereof. One skilled in the relevant art will also recognize that the technology can be practiced without one or more of the specific details, or with other methods, components, compounds, ingredients, etc. In other instances, well-known materials, or operations may not be shown or described in detail to avoid obscuring aspects of the disclosure.

In this application, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open-ended term in this written description, like “comprising” or “including,” it is understood that direct support should be afforded also to “consisting essentially of” language as well as “consisting of” language as if stated explicitly and vice versa.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a given term, metric, value, range endpoint, or the like. The degree of flexibility for a particular variable can be readily determined by one skilled in the art. However, unless otherwise expressed, the term “about” generally provides flexibility of less than 0.01%. It is to be understood that, even when the term “about” is used in the present specification in connection with a specific numerical value, support for the exact numerical value recited apart from the “about” terminology is also provided.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 1.5, 2, 2.3, 3, 3.8, 4, 4.6, 5, and 5.1 individually. This same principle applies to ranges reciting only one numerical value as a minimum or a maximum. Furthermore, such an interpretation should apply regardless of the breadth of the range or the characteristics being described.

Reference throughout this specification to “an example” means that a particular feature, structure, or characteristic described in connection with the example is included in at least one embodiment. Thus, appearances of phrases including “an example” or “an embodiment” in various places throughout this specification are not necessarily all referring to the same example or embodiment.

As used herein, the term “animal” refers to a non-human animal, such as mammals, aves, marsupials, and the like. The term “animal” includes reference to livestock, pets, exotic animals, racehorses and race dogs, rabbits, rodents, or any other animal cared for by humans or considered to have some value by humans.

As used herein, the term “target animal” refers to an animal being fed an animal nutritional product of the present disclosure.

Animals play an important role in the lives of many people, whether they be a pet, a food source, a means of entertainment, or the like. Additionally, most, if not all, animals can become sick from various diseases, the effects of malnutrition and/or neglect, auto-immune diseases, pathogenic infection, and cancer. Due to their importance, remedies for treating such sicknesses have been developed. As with humans, cancer is one condition that can be difficult to treat in animals. The present disclosure provides compositions, systems, and methods that can be used to not only treat many of these conditions, but also to serve as a prophylactic against them as well. While much of the following describes cancer treatment, such is for convenience, and it should be understood that any condition afflicting an animal that can be treated or otherwise alleviated according to the present disclosure is considered to be within the present scope.

As a more specific example, the present disclosure provides an animal treatment product that includes an animal food product and a cellular energy inhibitor composition that is combined with the animal product. Delivery of a therapeutic system to an animal such as a dog can be facilitated by mixing or otherwise combining the therapeutic composition with an animal food and/or food product. Additionally, such a food-based therapeutic provides an easily measured portion of the therapeutic composition at the proper dosage. In other examples, a therapeutic system can be delivered to an animal by traditional routes.

In addition, the method of administration can include inter-arterially, intravenously, inter-peritoneally, inhalation, intra-tumorally, orally, topically, and subcutaneously. In one embodiment, the administration can be inter-arterially. The compositions can also be delivered by using a feeding tube. Intra-tumoral delivery methods can include technologies involving a bronchoscope, an endoscope, and/or a colonoscopy, suppository to any openings, eye drops, nose drops, and ear drops. In one embodiment, the administration can be by intranasal delivery.

Intranasal delivery can be used to bypass the blood brain barrier and can be particularly effective for tumors in the brain and/or spinal cord. In another embodiment, the administration can be by suppository. Suppository administration can be used for tumors in proximity to the rectal/anal area. Additionally, if intra-tumoral injection is to be performed directly to/into the tumor, ultrasound imaging (or other imaging methods) can be used to aid this injection. In one embodiment, the administration can be by direct injection; e.g., to a prostate gland. Additionally, administration can be by an enema containing the composition described herein into the rectum and/or lower intestines. Chronic irrigation to treat obstructive colon, intestinal, or other obstructive cancers, can also be used in conjunction with the compositions described herein. In one embodiment, administration can also be by catheter to treat bladder cancers via the urethra. Further, intra venous delivery can be combined with a hemodialysis to destroy the metastatic circulating cancer cells outside of the blood vessels. In addition, both intravenous and inter-peritoneal can be assisted by utilization of a port system. Furthermore, the present compositions can be immediate release, controlled release, or time-controlled release. For time controlled release, the present compositions can be delivered by implanting wafers, diamond chips, and other implantable devices near or on the tumor site.

Generally, when the anti-cancer composition is administered intra-arterially or intravenously, the administration can be for a duration from about 30 minutes to about 8 hours. In one embodiment, the composition can be intraarterially or intravenously administered for a duration from about 3 hours to about 5 hours. Additionally, the administration of the anti-cancer composition can be part of a dosing regimen. In one embodiment, the administration can include a regimen lasting from about 1 week to 24 weeks. In another embodiment, the regimen can last from about 4 weeks to 8 weeks or more.

While some newer targeted therapies and immunotherapies have shown some success, many cancer and disease types don't respond well to treatment and can cause significant short- and long-term toxicities to animals. Cellular energy inhibitor formulations of the present disclosure effectively treat many of the aforementioned conditions with a low toxicity to the animal, particularly when compared to other treatments. One nonlimiting example of a cellular energy inhibitor is 3-bromopyruvic acid and its salt 3-bromopyruvate (referred to herein collectively as 3-BP).

In general, 3-BP is effective in modulating or otherwise eliminating abnormal and/or pathological cells such as cancer cells and pathogenic organisms, and thus can be used as an anti-cancer, anti-viral, anti-bacterial, anti-fungal, and anti-parasitic agent. For example, 3-BP effectively treats many cancers by targeting the metabolic energy production mechanisms in cancer cells. While energy metabolism reactions of eukaryotic cells are quite complex, two of the primary cellular energy production mechanisms are glycolysis, which occurs in the cytosol and oxidative phosphorylation, which occurs in the mitochondria. In the cytosol, glucose is split into pyruvate under aerobic conditions and lactate under anaerobic conditions. Under aerobic conditions, glycolysis converts one molecule of glucose into two molecules of pyruvate (pyruvic acid), generating energy in the form of adenosine triphosphate (ATP), a molecule that provides energy to the cell. In normal cells, a small proportion of the total ATP production is derived from glycolysis, with a significant majority of ATP being produced via oxidative phosphorylation in the mitochondria. In cancer cells, on the other hand, energy production in the cytosol via glycolysis can be significantly upregulated, which results in a significant increase in ATP production, up to 60% or more in some cases.

Many types of cancers exhibit this significant increase in glycolysis, known as the “Warburg Effect”, which is a common metabolic phenotype. Cancer cells that exhibit the Warburg effect increase lactic acid production as a result, which is transported out of the cells via monocarboxylate transporters (MCTs). MCTs are a family of plasma membrane proteins that transport molecules having one carboxylate group, such as lactate, pyruvate, and ketones, for example, across biological membranes. While normal cells express MCTs, cancer cells express MCTs in significantly greater numbers due to increased glycolysis generating greater amounts of lactic acid.

Cellular energy inhibitors of the present disclosure have chemical structures that are sufficiently similar to lactic acid to enter cancer cells via MCTs. Such cellular energy inhibitors are taken up by normal cells to a much smaller extent, if at all, due to the significantly lower numbers of MSCs. Due to a cellular energy inhibitor's highly reactive nature, it inactivates the glycolysis and oxidative phosphorylation systems, thus disrupting the energy production of the cancer cell and rapidly depleting ATP. Without cellular energy in the form of ATP, the cancer cells begin to die.

Cancers can be especially troubling in canines. More than 4 million dogs will be diagnosed with cancer this year, with only about one million undergoing a therapeutic treatment. Cancer is a major health problem in dogs, resulting in approximately 30% of total canine deaths, and is one of the leading causes of death for dogs over 6 years of age.

For pet dogs, the most common treatment options are surgery, radiotherapy, chemotherapy, and the like. For sarcomas and anal sac adenocarcinoma, aggressive surgical resection with wide margins is recommended, which can lead to permanent disfigurement or amputation, or in the case of ASAC can lead to fecal incontinence and nerve damage. Both of these aggressive canine cancers typically recur after surgery and metastasize to local lymph nodes.

Furthermore, it is recommended that a dog undergo 50-40 days of post-excision radiation therapy, which is costly and can lead to a diminished life. Standard chemotherapy is generally advised, as it is not curative and tends to cause unwanted side effects. effects. As with older humans, surgical and radiation therapy anesthetics are risky for older pets. Rates of recurrence are high despite aggressive treatments, and overall survival is often short—about 6 months to about 1.5 years.

While many cancers afflict dogs, some more common types include, without limitation, canine spindle cell soft sarcoma, anal sac adenocarcinoma, lymphoma, bone tumors, mast cell tumors, oral tumors, nasal tumors, bladder cancers, hemangiosarcomas, liver cancers, thyroid cancers, stomach cancers, and the like.

Examples of cellular energy inhibitors include lactate, iodoacetate, pyruvate, and a halopyruvate, including salts and acids thereof. As described above, 3-BP is an example a useful cellular energy inhibitor. 3-BP is a small molecule is sufficiently similar in chemical structure to lactic acid to enter cancer cells through an upregulated MTC transport system. Once in a cancer cell, 3-BP damages glycolysis and oxidative phosphorylation systems due to its highly reactive nature, thus significantly reducing ATP production. This reduction in ATP production subsequently leads to the death of the cancer cell. As such, 3-BP has a direct therapeutic effect on cancer cells due to its ability to inhibit energy production inside these cells.

In one example, the cellular energy inhibitor can be a molecule according to Formula I.

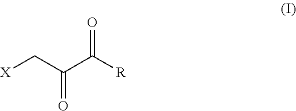

Various specific molecules are contemplated, wherein, for example, X can be, without limitation, a nitro, an imidazole, a halide, sulfonate, a carboxylate, an alkoxide, amine oxide, or the like. Additionally, R can be, without limitation, OR′, N(R″)₂, C(O)R″′, C1-C6 alkyl, C6-C12 aryl, C1-C6 heteroalkyl, a C6-C12 heteroaryl, O⁽⁻⁾, H, an alkali metal or the like, where R′ represents H, alkali metal, C1-C6 alkyl, C6-C12 aryl or C(O)R″′, R″ represents H, C1-C6 alkyl, or C6-C12 aryl, and R″′ represents H, C1-C20 alkyl or C6-C12 aryl.

In some examples, a cellular energy inhibitor composition can include a variety of excipients, active agents, prodrugs, metabolites, buffers, and the like, such as, for example, one or more sugars, polyalcohols, or the like, glycolysis inhibitors, biological buffers, or the like. In some examples the cellular energy inhibitor molecule can be formulated in a composition with at least one sugar, which can stabilize the cellular energy inhibitor by substantially preventing the cellular energy inhibitor from hydrolyzing.

In one example, R of formula (I) can be OH or O⁽⁻⁾ and X of formula (I) can be a nitro, an imidazole, a halide, a sulfonate, a carboxylate, an alkoxide, an amine oxide, or the like. Additionally, X can be a halide, such as, for example, fluoride, bromide, chloride, iodide, or the like. In one example, X can be a sulfonate, such as, for example, a triflate, a mesylate, a tosylate, or the like. In another example, X can be amine oxide. In still another example, the amine oxide can be dimethylamine oxide.

In another example, the cellular energy inhibitor can be a 3-halopyruvate, such as, for example, 3-fluoropyruvate, 3-chloropyruvate, 3-bromopyruvate, 3-iodopyruvate, or a combination thereof. A general structure showing a halide in the 3-position is shown in formula II.

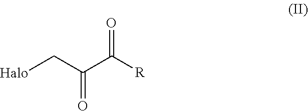

In a further nonlimiting example, the cellular energy inhibitor can have bromine in the 3-position, as shown in formula III.

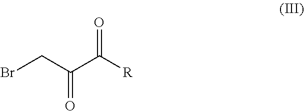

In one further nonlimiting example, the R of the cellular energy inhibitor of formula III can be O⁽⁻⁾, (3-bromopyruvate), as shown in formula IV.

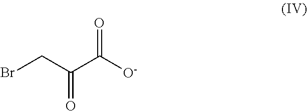

In another nonlimiting example, the R of the cellular energy inhibitor of formula III can be OH (3-bromopyruvic acid), as shown in formula V.

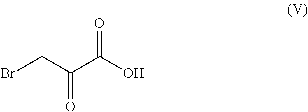

It is noted that 3-bromopyruvate or 3-bromopyruvic acid can be referred to herein as “3-BP,” and that the two molecules can be used interchangeably unless the context clearly indicates otherwise.

Generally, 3-BP can be formulated as any type of dosage form capable of being delivered to a subject. Such dosage forms can be enteral, parenteral, transdermal, or the like. Enteral dosage forms can be sustained release or immediate release and can include, without limitation, tablets, lozenges, capsules, caplets, encapsulated pellets, encapsulated granules, encapsulated powders, gelatin capsules, liquids, syrups, elixirs, suspensions, sprays, aerosols, powders, and the like, including combinations thereof. Nonlimiting examples of transdermal dosage forms can include lotions, gels, creams, pastes, ointments, liquid sprays, liquid drops, powder sprays, wipes, emulsions, aerosols, transmucosal tablets, adhesive devices, adhesive matrix-type transdermal patches, liquid reservoir transdermal patches, microneedle devices, magnetic devices, and the like. Nonlimiting examples of parenteral dosage forms can include intravenous, subcutaneous, and the like.

In one example, 3-BP compositions of the present disclosure can be formulated in a composition with at least one sugar, which can stabilize the 3-BP molecule by substantially preventing it from hydrolyzing. In other examples, s 3-BP composition includes multiple sugars to stabilize the 3-BP molecule. The multiple sugars can include at least two sugars, at least three sugars, and so on.

While various sugars are contemplated, in some examples a sugar can include a monosaccharide, a disaccharide, an oligosaccharide, or a combination thereof. Nonlimiting examples of monosaccharides can include glucose, fructose, galactose, and the like. Nonlimiting examples of disaccharides can sucrose, lactose, maltose, and the like. It is noted that, for the purposes of the present disclosure, the term “sugar” can also include oligosaccharides, polysaccharides, polyols, polyalcohols, and similar molecules, such as glycerol, that function to stabilize 3-BP. A sugar can include a 3-carbon sugar, a 4-carbon sugar, a 5-carbon sugar, a 6-carbon sugar, a 7-carbon sugar, and the like, including combinations thereof. In one aspect, the sugar can be a nonmetabolizable.

In one example, the sugar is gluconic acid. In another example, the sugar is glucuronic acid. In another example, at least one of sugar is a five-carbon sugar. In another example, at least two sugars are five-carbon sugars. The five-carbon sugars can be independently selected from mannitol, erythritol, isomalt, lactitol, maltitol, sorbitol, xylitol, dulcitol, ribitol, inositol, or the like, including combinations thereof. In another example, at least one of sugar can be glycerol. In another example, the sugar is glycerol, inositol, and sorbitol. Other nonlimiting example of sugars can include ethylene glycol, threitol, arabitol, galactitol, fucitol, iditol, volemitol, maltotriitol, maltotetraitol, and polyglycitol, including combinations thereof. In one example, the sugar includes glycerol, inositol, sorbitol, mannitol or any combination thereof. In another example, the sugar includes glycerol, inositol, sorbitol, or any combination thereof. In other examples, the sugar is a polyalcohol.

The sugars described herein can be any isomeric form. In one example, the compositions described herein the less biologically active form of the sugar as compared to its isomer. In one case, the less biologically active sugar can be the L-enantiomer sugar. However, if the D-enantiomer sugar is found to be less biologically active as compared to its L form, then the D form can be used. In one example, such sugars can function as a glycolytic inhibitor.

In one example, a composition includes one or more sugars in a range from about 0.5 wt % to about 50.0 wt % or from about 1.0 wt % to about 25.5 wt %. In yet another example, a composition includes one or more sugars in a range from about 0.2 wt % to about 75.0 wt % or from about 0.5 wt % to about 50.0 wt %. In a further example, a composition includes one or more sugars in a range from about 0.1 wt % to about 25.0 wt %, from about 0.2 wt % to about 10.0 wt %.

In some examples, the composition includes glycerol in a range from about 0.1 wt % to about 5.0 wt % or from about 0.1 wt % to about 3.0 wt %. In other examples, the composition includes inositol in a range from about 0.1 wt % to about 10 wt %, from about 0.1 wt % to about 6 wt %. In further examples, the composition includes sorbitol in a range from about 0.1 wt % to about 40.0 wt % or from about 0.1 wt % to about 30 wt %. In yet further examples, the composition includes mannitol in a range from about 0.1 wt % to about 30 wt % or from about 0.1 wt % to about 10 wt %. Additionally, each of the sugars may be added in a volume up to a maximum solubility of the sugar in the formulation or composition. It is additionally noted that the above wt % s of ingredients are without water or other liquid carrier. Additionally, each of the sugars may be added in a volume up to a maximum solubility of the sugar in the formulation or composition.

A 3-BP composition can include a biological buffer that is present in an amount sufficient to at least partially deacidify the cellular energy inhibitor and neutralize metabolic by-products of the cellular energy inhibitor. Nonlimiting examples of biological buffers can include a citrate buffer, a phosphate buffer, an acetate buffer, and the like, including combinations thereof. In one specific example, the biological buffer can be a citrate buffer, such as, without limitation, sodium citrate. In another specific example, the biological buffer can be a phosphate buffer, such as, without limitation, sodium phosphate. In one specific example, the biological buffer can be an acetate buffer, such as, without limitation, sodium acetate. In yet other examples, the biological buffer can include at least two biological buffers, such as, without limitation, a citrate buffer and an acetate buffer, a citrate buffer and a phosphate buffer, an acetate buffer and a phosphate buffer, or a citrate buffer, a phosphate buffer, and an acetate buffer.

In some examples, the composition can comprise the biological buffer in a concentration of from about 0.1 mM to about 200 mM. In one embodiment, the composition can comprise the biological buffer in a concentration of from about 1 mM to about 20 mM. In some examples, the composition can include the biological buffer in a range of from about 0.1 wt % to about 15 wt % or from about 2.0 wt % to about 8.0 wt %. Additionally, the biological buffer can maintain a physiological pH of 4.0 to 8.5. In one embodiment, the biological buffer can maintain a physiological pH of 5.5 to 8.0. In another embodiment, the biological buffer can maintain a physiological pH of 6.8 to 7.8. In still another embodiment, the biological buffer can maintain a physiological pH of 7.3 to 7.6. It is additionally noted that the above wt % s of ingredients are without water or other liquid carrier.

In some examples, the present 3-BP formulations can comprise antifungal agents, antibiotics, glycolysis inhibitors, inhibitors of mitochondria, sugars, and biological buffers, without limitation. Examples of such agents include, but are not limited to, amphotericin B, efrapeptin, doxorubicin, (2-DG), analogs of 2-DG, d-lactic acid, dichloroacetic acid (or salt form of dichloroacetate), oligomycin, analogs of oligomycin, glycerol, inositol, sorbitol, glycol, erythritol, threitol, arabitol, xylitol, ribitol, mannitol, dulcitol, iditol, isomalt, maltitol, lactitol, polyglycitol, sodium phosphate, sodium citrate, sodium acetate, sodium carbonate, sodium bicarbonate, sodium pyruvate, sodium lactate, oxaloacetate, isocitrate, aconitate, succinate, fumarate, malate, diluted saline solutions with varying concentrations of NaCl, and water. In addition to the sodium ion that accompanies these biological buffers, calcium and potassium cations can also accompany the biological buffers. Various active agents of the composition can include a cellular energy inhibitor, a glycolysis inhibitor, a mitochondria inhibitor, a halo monocarboxylate compound, an antifungal agent, an antibiotic agent, and the like. In the various dosage forms described above, any of the above ingredients can be included with 3-BP, any of the excipients, or in a separate vessel.

In addition to the above components, the 3-BP compositions described herein can further comprise a halo monocarboxylate compound that is separate from the cellular energy inhibitor. In cases where the halo monocarboxylate compound can function to inhibit glycolysis and/or mitochondria function, the halo monocarboxylate can be considered a second cellular energy inhibitor. In one embodiment, the halo monocarboxylate compound can be a halo two-carbon monocarboxylate compound. The halo two-carbon monocarboxylate compound can be selected from, without limitation, 2-fluoroacetate, 2-chloroacetate, 2-bromoacetate, 2-iodoacetate, and the like, including combinations thereof. In one embodiment, the halo two-carbon monocarboxylate compound can be 2-bromoacetate. In one example, the composition can comprise the halo two-carbon monocarboxylate compound in a concentration from about 0.01 mM to about 5.0 mM. In another example, the composition can comprise a halo two-carbon monocarboxylate compound in a concentration from about 0.1 mM to about 0.5 mM.

Additionally, a halo monocarboxylate compound can be a halo three-carbon monocarboxylate compound. In one embodiment, the halo three-carbon monocarboxylate compound can be selected from, without limitation, 3-fluorolactate, 3-chlorolactate, 3-bromolactate, 3-iodolactate, and the like, including combinations thereof. In another example, the composition can include the halo three-carbon monocarboxylate compound in a concentration from about 0.5 mM to about 250 mM. In one embodiment, the composition can comprise the halo three-carbon monocarboxylate compound in a concentration from about 10 mM to about 50 mM.

In some examples, the 3-BP formulations described herein can further comprise a mitochondrial inhibitor in addition to the cellular energy inhibitor. The mitochondrial inhibitor can be selected from, without limitation, oligomycin, efrapeptin, aurovertin, and the like, including combinations thereof. In another example, the composition can include the mitochondrial inhibitor in a concentration from about 0.001 mM to about 5.0 mM. In one example, the composition can include the mitochondrial inhibitor in a concentration from about 0.01 mM to about 0.5 mM.

In some examples, the present 3-BP compositions described herein can further comprise an antifungal agent and/or antibacterial agent. In one embodiment, the composition can individually comprise the antifungal agent and/or antibacterial agent in a concentration from about 0.01 mM to about 5.0 mM. In another embodiment, the composition can individually comprise the antifungal agent and/or antibacterial agent in a concentration from about 0.05 mM to about 0.5 mM.

In some examples, a 3-BP formulation can include a glycolysis inhibitor. Many suitable glycolysis inhibitors are contemplated, however a nonlimiting list can include 2-DG, lonidamine, imatinib, oxythiamine, 6-aminonicotinamide, genistein, 5-thioglucose (5-TG), mannoheptulose, α-chlorohydrin, ornidazole, oxalate, glufosfamide, and the like, including combinations thereof. The 3-BP formulation can include the glycolysis inhibitor in any effective amount.

In addition to the above concentrations, the present compositions can have various ratios of the components described herein. In one embodiment, the cellular energy inhibitor and biological buffer can be present in a ratio ranging from 1:1 to 1:5 by mM. In another embodiment, the cellular energy inhibitor and glycolysis inhibitor can be present in a ratio ranging from 5:1 to 1:1 by mM. In still another embodiment, the cellular energy inhibitor and the at least one sugar are present in a ratio ranging from 1:1 to 1:5 by mM. In yet another embodiment, the cellular energy inhibitor and the halo two-carbon monocarboxylate compound can be present in a ratio ranging from 20:1 to 4:1 by mM. In still yet another embodiment, the cellular energy inhibitor to mitochondrial inhibitor can be present in a ratio ranging from 20:1 to 40:1 by mM.

In some examples, the 3-BP compositions described herein can further include a hexokinase inhibitor. The hexokinase inhibitor can be any molecule that inhibits hexokinase 1, hexokinase 2, and/or any isozyme thereof (collectively referred to herein as “hexokinase”). As used herein, “hexokinase 1” or “hexokinase 1 isozyme” refers to any isoforms of hexokinase 1 and its naturally known variants, including those provided in SEQ ID NO: 1, SEQ ID NO: 2, SEQ ID NO: 3, and SEQ ID NO: 4, as follows:

(SEQ ID NO: 1)

| | |
|---|---|
| 1 | MIAAQLLAYY FTELKDDQVK KIDKYLYAMR LSDETLIDIM TRFRKEMKNG LSRDFNPTAT |
| 61 | VKMLPTFVRS IPDGSEKGDF IALDLGGSSF RILRVQVNHE KNQNVHMESE VYDTPENIVH |
| 121 | GSGSQLFDHV AECLGDFMEK RKIKDKKLPV GFTFSFPCQQ SKIDEAILIT WTKRFKASGV |
| 181 | EGADVVKLLN KAIKKRGDYD ANIVAVVNDT VGTMMTCGYD DQHCEVGLII GTGTNACYME |
| 241 | ELRHIDLVEG DEGRMCINTE WGAFGDDGSL EDIRTEFDRE IDRGSLNPGK QLFEKMVSGM |
| 301 | YLGELVRLIL VKMAKEGLLF EGRITPELLT RGKFNTSDVS AIEKNKEGLH NAKEILTRLG |
| 361 | VEPSDDDCVS VQHVCTIVSF RSANLVAATL GAILNRLRDN KGTPRLRTTV GVDGSLYKTH |
| 421 | PQYSRRFHKT LRRLVPDSDV RFLLSESGSG KGAAMVTAVA YRLAEQHRQI EETLAHFHLT |
| 481 | KDMLLEVKKR MRAEMELGLR KQTHNNAVVK MLPSFVRRTP DGTENGDFLA LDLGGTNFRV |
| 541 | LLVKIRSGKK RTVEMHNKIY AIPIEIMQGT GEELFDHIVS CISDFLDYMG IKGPRMPLGF |
| 601 | TFSFPCQQTS LDAGILITWT KGFKATDCVG HDVVTLLRDA IKRREEFDLD VVAVVNDTVG |
| 661 | TMMTCAYEEP TCEVGLIVGT GSNACYMEEM KNVEMVEGDQ GQMCINMEWG AFGDNGCLDD |
| 721 | IRTHYDRLVD EYSLNAGKQR YEKMISGMYL GEIVRNILID FTKKGFLFRG QISETLKTRG |
| 781 | IFETKFLSQI ESDRLALLQV RAILQQLGLN STCDDSILVK TVCGVVSRRA AQLCGAGMAA |
| 841 | VVDKIRENRG LDRLNVTVGV DGTLYKLHPH FSRIMHQTVK ELSPKCNVSF LLSEDGSGKG |
| 901 | AALITAVGVR LRTEASS |

(SEQ ID NO: 2)

| | |
|---|---|
| 1 | MDCEHSLSLP CRGAEAWEIG IDKYLYAMRL SDETLIDIMT RFRKEMKNGL SRDFNPTATV |
| 61 | KMLPTFVRSI PDGSEKGDFI ALDLGGSSFR ILRVQVNHEK NQNVHMESEV YDTPENIVHG |
| 121 | SGSQLFDHVA ECLGDFMEKR KIKDKKLPVG FTFSFPCQQS KIDEAILITW TKRFKASGVE |
| 181 | GADVVKLINK AIKKRGDYDA NIVAVVNDTV GTMMTCGYDD QHCEVGLIIG TGTNACYMEE |
| 241 | LRHIDLVEGD EGRMCINTEW GAFGDDGSLE DIRTEFDREI DRGSLNPGKQ LFEKMVSGMY |
| 301 | LGELVRLILV KMAKEGLLFE GRITPELLTR GKFNTSDVSA IEKNKEGLHN AKEILTRLGV |
| 361 | EPSDDDCVSV QHVCTIVSFR SANLVAATLG AILNRLRDNK GTPRLRTTVG VDGSLYKTHP |
| 421 | QYSRRFHKTL RRLVPDSDVR FLLSESGSGK GAAMVTAVAY RLAEQHRQIE ETLAHFHLTK |
| 481 | DMLLEVKKRM RAEMELGLRK QTHNNAVVKM LPSFVRRTPD GTENGDFLAL DLGGTNFRVL |
| 541 | LVKIRSGKKR TVEMHNKIYA IPIEIMQGTG EELFDHIVSC ISDFLDYMGI KGPRMPLGFT |
| 601 | FSFPCQQTSL DAGILITWTK GFKATDCVGH DVVTLLRDAI KRREEFDLDV VAVVNDTVGT |
| 661 | MMTCAYEEPT CEVGLIVGTG SNACYMEEMK NVEMVEGDQG QMCINMEWGA FGDNGCLDDI |
| 721 | RTHYDRLVDE YSLNAGKQRY EKMISGMYLG EIVRNILIDF TKKGFLFRGQ ISETLKTRGI |
| 781 | FETKELSQIE SDRLALLQVR AILQQLGINS TCDDSILVKT VCGVVSRRAA QLCGAGMAAV |
| 841 | VDKIRENRGL DRLNVTVGVD GTLYKLHPHF SRIMHQTVKE LSPKCNVSFL LSEDGSGKGA |
| 901 | ALITAVGVRL RTEASS |

(SEQ ID NO: 3)

| | |
|---|---|
| 1 | MGQICQRESA TAAEKPKLHL LAESEIDKYL YAMRLSDETL IDIMTRFRKE MKNGLSRDEN |
| 61 | PTATVKMLPT FVRSIPDGSE KGDFIALDLG GSSFRILRVQ VNHEKNQNVH MESEVYDTPE |
| 121 | NIVHGSGSQL FDHVAECLGD FMEKRKIKDK KLPVGFTFSF PCQQSKIDEA ILITWTKRFK |
| 181 | ASGVEGADVV KLLNKAIKKR GDYDANIVAV VNDTVGTMMT CGYDDQHCEV GLIIGTGTNA |
| 241 | CYMEELRHID LVEGDEGRMC INTEWGAFGD DGSLEDIRTE FDREIDRGSL NPGKQLFEKM |
| 301 | VSGMYLGELV RLILVKMAKE GLLFEGRITP ELLTRGKENT SDVSAIEKNK EGLHNAKEIL |
| 361 | TRLGVEPSDD DCVSVQHVCT IVSFRSANLV AATLGAILNR LRDNKGTPRL RTTVGVDGSL |
| 421 | YKTHPQYSRR FHKTLRRLVP DSDVRELLSE SGSGKGAAMV TAVAYRLAEQ HRQIEETLAH |
| 481 | FHLTKDMLLE VKKRMRAEME LGLRKQTHNN AVVKMLPSFV RRTPDGTENG DFLALDLGGT |
| 541 | NFRVLLVKIR SGKKRTVEMH NKIYAIPIEI MQGTGEELFD HIVSCISDEL DYMGIKGPRM |
| 601 | PLGFTFSFPC QQTSLDAGIL ITWTKGFKAT DCVGHDVVTL LRDAIKRREE FDLDVVAVVN |
| 661 | DTVGTMMTCA YEEPTCEVGL IVGTGSNACY MEEMKNVEMV EGDQGQMCIN MEWGAFGDNG |
| 721 | CLDDIRTHYD RLVDEYSLNA GKQRYEKMIS GMYLGEIVRN ILIDFTKKGF LFRGQISETL |
| 781 | KTRGIFETKF LSQIESDRLA LLQVRAILQQ LGLNSTCDDS ILVKTVCGVV SRRAAQLCGA |
| 841 | GMAAVVDKIR ENRGLDRLNV TVGVDGTLYK LHPHFSRIMH QTVKELSPKC NVSELLSEDG |
| 901 | SGKGAALITA VGVRLRTEAS S |

(SEQ ID NO: 4)

| | |
|---|---|
| 1 | MAKRALRDFI DKYLYAMRLS DETLIDIMTR FRKEMKNGLS RDENPTATVK MLPTFVRSIP |
| 61 | DGSEKGDFIA LDLGGSSFRI LRVQVNHEKN QNVHMESEVY DTPENIVHGS GSQLFDHVAE |
| 121 | CLGDFMEKRK IKDKKLPVGF TFSFPCQQSK IDEAILITWT KRFKASGVEG ADVVKLLNKA |
| 181 | IKKRGDYDAN IVAVVNDTVG TMMTCGYDDQ HCEVGLIIGT GTNACYMEEL RHIDLVEGDE |
| 241 | GRMCINTEWG AFGDDGSLED IRTEFDREID RGSLNPGKQL FEKMVSGMYL GELVRLILVK |
| 301 | MAKEGLLFEG RITPELLTRG KENTSDVSAI EKNKEGLHNA KEILTRLGVE PSDDDCVSVQ |
| 361 | HVCTIVSFRS ANLVAATLGA ILNRLRDNKG TPRLRTTVGV DGSLYKTHPQ YSRRFHKTLR |
| 421 | RLVPDSDVRF LLSESGSGKG AAMVTAVAYR LAEQHRQIEE TLAHFHLTKD MLLEVKKRMR |
| 481 | AEMELGLRKQ THNNAVVKML PSFVRRTPDG TENGDFLALD LGGTNFRVLL VKIRSGKKRT |
| 541 | VEMHNKIYAI PIEIMQGTGE ELFDHIVSCI SDELDYMGIK GPRMPLGFTF SFPCQQTSLD |
| 601 | AGILITWTKG FKATDCVGHD VVTLLRDAIK RREEFDLDVV AVVNDTVGTM MTCAYEEPTC |
| 661 | EVGLIVGTGS NACYMEEMKN VEMVEGDQGQ MCINMEWGAF GDNGCLDDIR THYDRLVDEY |
| 721 | SLNAGKQRYE KMISGMYLGE IVRNILIDFT KKGFLERGQI SETLKTRGIF ETKELSQIES |
| 781 | DRLALLQVRA ILQQLGLNST CDDSILVKTV CGVVSRRAAQ LCGAGMAAVV DKIRENRGLD |
| 841 | RLNVTVGVDG TLYKLHPHFS RIMHQTVKEL SPKCNVSFLL SEDGSGKGAA LITAVGVRLR |
| 901 | TEASS |

As used herein, “hexokinase 2” or “hexokinase 2 isozyme” refers to any isoforms of hexokinase 2 and its naturally known variants, including that provided in SEQ ID NO: 5 as follows:

| (SEQ ID NO: 5) | |
|---|---|
| 1 | MIASHLLAYF FTELNHDQVQ KVDQYLYHMR LSDETLLEIS KRFRKEMEKG LGATTHPTAA |
| 61 | VKMLPTFVRS TPDGTEHGEF LALDLGGTNF RVLWVKVTDN GLQKVEMENQ IYAIPEDIMR |
| 121 | GSGTQLFDHI AECLANFMDK LQIKDKKLPL GFTFSFPCHQ TKLDESFLVS WTKGFKSSGV |
| 181 | EGRDVVALIR KAIQRRGDFD IDIVAVVNDT VGTMMTCGYD DHNCEIGLIV GTGSNACYME |
| 241 | EMRHIDMVEG DEGRMCINME WGAFGDDGSL NDIRTEFDQE IDMGSLNPGK QLFEKMISGM |
| 301 | YMGELVRLIL VKMAKEELLF GGKLSPELLN TGRFETKDIS DIEGEKDGIR KAREVLMRLG |
| 361 | LDPTQEDCVA THRICQIVST RSASLCAATL AAVLQRIKEN KGEERLRSTI GVDGSVYKKH |
| 421 | PHFAKRLHKT VRRLVPGCDV RFLRSEDGSG KGAAMVTAVA YRLADQHRAR QKTLEHLQLS |
| 481 | HDQLLEVKRR MKVEMERGLS KETHASAPVK MLPTYVCATP DGTEKGDFLA LDLGGTNFRV |
| 541 | LLVRVRNGKW GGVEMHNKIY AIPQEVMHGT GDELFDHIVQ CIADFLEYMG MKGVSLPLGF |
| 601 | TFSFPCQQNS LDESILLKWT KGFKASGCEG EDVVTLLKEA IHRREEFDLD VVAVVNDTVG |
| 661 | TMMTCGFEDP HCEVGLIVGT GSNACYMEEM RNVELVEGEE GRMCVNMEWG AFGDNGCLDD |
| 721 | FRTEFDVAVD ELSLNPGKQR FEKMISGMYL GEIVRNILID FTKRGLLFRG RISERLKTRG |
| 781 | IFETKFLSQI ESDCLALLQV RAILQHLGLE STCDDSIIVK EVCTVVARRA AQLCGAGMAA |
| 841 | VVDRIRENRG LDALKVTVGV DGTLYKLHPH FAKVMHETVK DLAPKCDVSF LQSEDGSGKG |
| 901 | AALITAVACR IREAGQR |

As has been described, a major source of ATP production occurs in mitochondria in normal cells. However, ATP production from glycolysis is significantly upregulated in cancer cells. One reason for this upregulation is due to hexokinase molecules binding to, and forming complexes with, mitochondrial voltage dependent anion channels (VDACs) at ATP synthasomes, thus forming so called “ATP synthasome mega complexes.” The formation of such ATP synthasome mega complexes can immortalize the cancer cell, thus allowing the continued use of the cell's energy production processes for cancer growth. A hexokinase inhibitor, therefore, can thus block hexokinase from binding to the VADCs or displace hexokinase molecules from the VADCs of already formed ATP synthasome mega complexes.

In one example, a hexokinase inhibitor can be up to 25 amino acid units from the N-terminal region of Hexokinase 2 isozyme or Hexokinase 1 isozyme. In another example, the hexokinase inhibitor can be an amino acid sequence of 5 to 20 amino acid units, where the 5 to 20 amino acid sequence is present in the first 25 amino acid unit region beginning from the N-terminal end of hexokinase 1 isozyme or hexokinase 2 isozyme. In one example, the 5 to 20 amino acid sequence can be any 5-20 amino acid sequence present in the first 25 amino acid unit region of the N-terminus of Hexokinase 1 1 or Hexokinase 2. Such amino acid sequences can displace cellular bound hexokinase or competitively bind to voltage dependent anion channels (VDAC), thus preventing initial hexokinase binding.

In other examples, a hexokinase inhibitor can include antibodies against a portion of HK1 or HK2, such as, for example, the N-terminal region of either molecule. In one specific example, a hexokinase inhibitor can be an amino acid sequence, such as SEQ ID NO: 6, corresponding to the first 25 amino acids from the N-terminus end of hexokinase 1 (isoform1) having a sequence as follows:

| (SEQ ID NO: 6) | |
|---|---|
| 1 | MIAAQLLAYY FTELKDDQVK KIDKY |

In another example, a hexokinase inhibitor can be an amino acid sequence as in SEQ ID NO: 7, corresponding to the first 25 amino acids from the N-terminus end of hexokinase 1 (isoform 2) having a sequence as follows:

| (SEQ ID NO: 7) | |
|---|---|
| 1 | MDCEHSLSLP CRGAEAWEIG IDKYL |

In yet another example, a hexokinase inhibitor can be an amino acid sequence as in SEQ ID NO: 8, corresponding to the first 25 amino acids from the N-terminus end of hexokinase 1 (isoform 3) having a sequence as follows:

| (SEQ ID NO: 8) | |
|---|---|
| 1 | MGQICQRESA TAAEKPKLHL LAESE |

In still another example, a hexokinase inhibitor can be an amino acid sequence as in SEQ ID NO: 9, corresponding to the first 25 amino acids from the N-terminus end of hexokinase 1 (isoform 4) having a sequence as follows:

| (SEQ ID NO: 9) | |
|---|---|
| 1 | MAKRALRDFI DKYLYAMRLS DETLI |

In yet another example, a hexokinase inhibitor can be an amino acid sequence as in SEQ ID NO: 10, corresponding to the first 25 amino acids from the N-terminus end of hexokinase 2 having a sequence as follows:

(SEQ ID NO: 10)
MIASHLLAYF FTELNHDQVQ KVDQY

Additional hexokinase inhibitors can be those as disclosed in U.S. Pat. No. 5,854,067 (to Newgard et al, issued Dec. 29, 1998) and/or U.S. Pat. No. 5,891,717 (to Newgard et al., issued Apr. 6, 1999), both of which are incorporated by reference in their entireties. Additional hexokinase inhibitors that can be used in the present formulations include those disclosed in U.S. Pat. Nos. 6,670,330; 6,218,435; 5,824,665; 5,652,273; and 5,643,883; and U.S. patent application publication Nos. 20030072814; 20020077300; and 20020035071; each of the foregoing patent publications and patent application is incorporated herein by reference, in their entireties.

In one embodiment, the present compositions can include less biologically active amino acids as compared to their isomers to facilitate cancer cell starvation. In one aspect, the less biologically active amino acid can be a D-amino acid. However, if the L-amino acid is less biologically active than the D-form, the L-amino acid can be used.

In one embodiment, the present compositions can include inhibitors for DNA replication; inhibitors for DNA binding; and/or inhibitors for DNA transcription. In another embodiment, the present compositions can include inhibitors for cell cycle, growth and/or proliferation. In yet another embodiment, the present compositions can include inhibitors for signal transduction pathways. In yet another embodiment, the present compositions can include inhibitors for angiogenesis. In yet another embodiment, the present compositions can include small RNAs that interfere with normal gene control including antisense RNA, micro RNA, small hairpin RNA, short hairpin RNA, small interfering RNA, and the like. In yet another embodiment, the present compositions can include vitamin C; nutritional supplements including vitamins, CoQ10, flavonoids, free fatty acid, alpha lipoic acid, acai, goji, mango, pomegranate, L-carnitine, selenium; etc.

In one example, a 3 composition can include 3-BP in the amount of from about 0.01 wt % to about 2.0 wt % from about 0.1 wt % to about 1.6 wt %.

The animal treatment product can be formed into a variety of shapes and sizes, which may in some cases depend on the preference of the animal or owner. In addition, the shape and/or size of a food product form can depend on the size and/or configuration of the animal's mouth. Nonlimiting examples can include food product forms such as patties, cubes, spheres, cylinders, discs, flakes, sheets, pellets, bone shapes, planar shapes, and the like.

As described above, the cellular energy inhibitor can be associated with the animal food ingredient in a variety of ways. In one specific example, the animal treatment product can include the animal food ingredient with a cellular energy inhibitor packaged and ready to be applied to the animal food ingredient. Maintaining the animal food ingredient separate from the cellular energy inhibitor can prolong the effectiveness of the cellular energy inhibitor. In the case of 3-BP formulations, for example, the 3-BP composition can be packaged such that the animal food ingredient and the 3-BP composition are not in contact with one another. In one example, the 3-BP composition and the animal food ingredient can be isolated from one another in the same package. To feed the animal treatment product to an animal, the owner can open the packaging, apply the 3-BP composition to the animal food ingredient, and feed the animal nutritional product to the target animal. Components of the 3-BP composition can be premixed together or packaged separately to be mixed by the owner. In some cases, one or more component can be premixed with the 3-BP and one or more components can be packaged separately. For example, in 3-BP compositions including a biological buffer, the 3-BP and sugars can be packaged together separately from the biological buffer. When ready to use, the owner can mix the buffer with the 3-BP and sugar mixture, apply the e-BP composition to the animal food ingredient and feed the resulting animal treatment product to the target animal.

In some examples, the 3-BP compositions described herein can further comprise various ingredients recited below. In the various dosage forms known to those skilled in the art, any of these various ingredients can be admixed with the 3-BP, provided they are nonreactive therewith, or be present in a separate layer or in any layer described above, provided the ingredient(s) is/are reactively isolated in the storage form.

As one example, an animal treatment product includes a food ingredient, which in some cases can be a majority food ingredient(s) (or food ingredient(s) that makes up a majority of the animal treatment product by weight), that can be appropriate for ingestion and digestion of a targeted given animal. The animal treatment product additionally includes a cellular energy inhibitor associated with the animal food ingredient. The cellular energy inhibitor is associated with the animal food ingredient at the time of manufacture of the animal food ingredient. Various techniques for associating the cellular energy inhibitor with the animal food ingredient are contemplated, such as, without limitation, mixing, layering, coating, encapsulating, sprinkling, spraying, and the like, including techniques combining the two or more of the aforementioned where appropriate.

The present disclosure provides animal treatment products, animal nutrition supplements for animal products, animal treatment systems, and various methods of use. Without intending to be bound to any scientific theory, such products, supplements, and systems can be used to treat an animal for a disease or other condition, be used as an adjuvant, be used as a prophylactic, or a combination thereof. Such effects can additionally include enhancement of related physiological systems, such as the immune system.

As one example, an animal treatment product includes a food ingredient, which in some cases can be a majority food ingredient(s) (or food ingredient(s) that makes up a majority of the animal treatment product by weight), that can be appropriate for ingestion and digestion of a targeted given animal. The animal treatment product additionally includes a cellular energy inhibitor associated with the animal food ingredient. The cellular energy inhibitor is associated with the animal food ingredient at the time of manufacture of the animal food ingredient. Various techniques for associating the cellular energy inhibitor with the animal food ingredient are contemplated, such as, without limitation, mixing, layering, coating, encapsulating, sprinkling, spraying, and the like, including techniques combining the two or more of the aforementioned where appropriate.

In another example, the cellular energy inhibitor can be associated with the animal food ingredient subsequent to manufacture of the animal food ingredient at some point along the distribution chain from the manufacturer to the animal owner, such as by a supplier or seller, for example. Various techniques for associating the cellular energy inhibitor with the animal food ingredient by entities along the distribution chain are contemplated, such as, without limitation, mixing, layering, coating, encapsulating, sprinkling, spraying, and the like, including techniques combining two or more of the aforementioned where appropriate. It is noted that the association techniques available to an entity would likely depend on the sophistication and/or technical resources available to that entity.

In yet another example, the cellular energy inhibitor can be associated with the animal food ingredient at the level of ownership and/or caretaking (hereinafter “ownership”) of the target animal. In such cases, the animal treatment product is obtained by the owner/caretaker (hereinafter “owner”) as a multicomponent product. Thus, the association of the cellular energy inhibitor with the animal food ingredient is affected by the owner physically combining the two ingredients together prior to feeding the target animal. The potential association techniques would depend on the sophistication and/or technical resources available to the owner, which could potentially include, without limitation, mixing, layering, coating, encapsulating, sprinkling, spraying, and the like, including techniques combining the two or more of the aforementioned where appropriate.

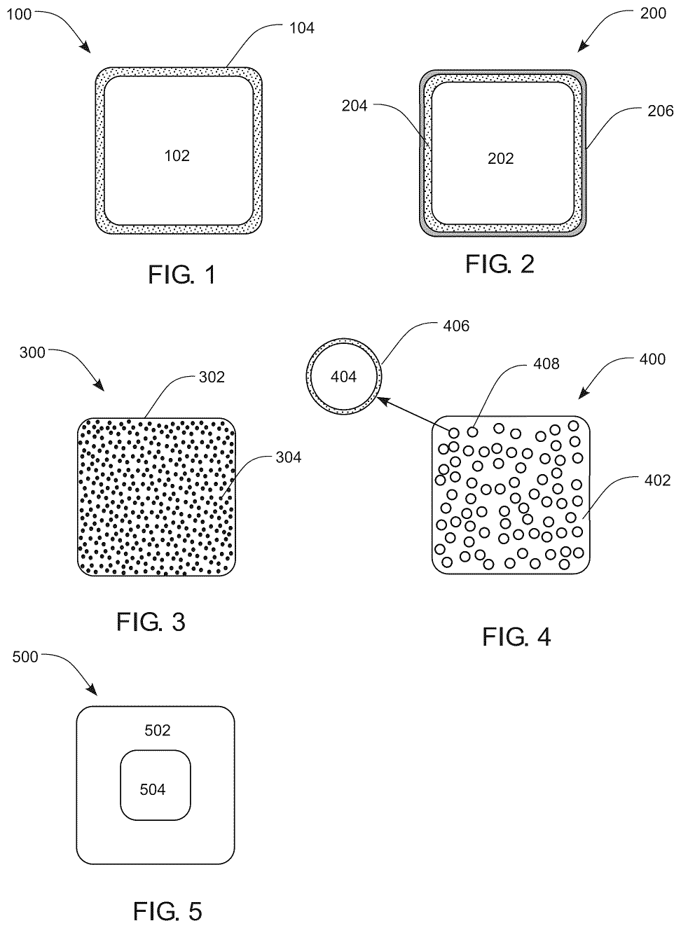

In one example, as is shown in FIG. 1, an animal treatment product 100 can include an animal food ingredient 102 surrounded by a layer or coating (hereinafter “layer”) of a cellular energy inhibitor 104. Depending on the nature of the cellular energy inhibitor and the design of the resulting animal treatment product, the layer or coating of the cellular energy inhibitor can be a solid layer, a semisolid layer, or the like. The layer can be continuous or discontinuous, and of any thickness that delivers an appropriate amount of the cellular energy inhibitor to the target animal. Regarding continuity, the layer can be a continuous layer across one or multiple sides/surfaces of the animal treatment product. A discontinuous layer can be a layer having gaps or substantially thinned portions across at least a portion of its surface. Sprinkling and light spraying, for example, can create a cellular energy inhibitor layer that is discontinuous or that has discontinuous portions.

In another example, as is shown in FIG. 2, an animal treatment product 200 can include an animal food ingredient 202 surrounded by multiple layers 204, 206. Layers 204 and 206 can each be separate components of a cellular energy inhibitor, or one of 204 or 206 can include the cellular energy inhibitor and the other can be a separate or nonimmune ingredient. Depending on the nature of the cellular energy inhibitor and the design of the resulting animal treatment product, the layers or coatings of the cellular energy inhibitor can be independently solid layers, a semisolid layer, or the like.

In a more specific example, the multiple layers 204 and 206 can be separate components of a cellular energy inhibitor. In one such configuration, the outer layer 206 can be an ingredient of the cellular energy inhibitor that is reactive with the animal food ingredient 202 and is thus separated therefrom by another ingredient of the cellular energy inhibitor that is nonreactive or less reactive compared to the outer layer ingredient. In another configuration, the ingredients of the cellular energy inhibitor may be reactive with one another, and as such, are maintained separately until broken up by the target animal. In such cases and third layer can also be disposed between the two layers 204 and 206 (not shown). In other configurations, the ingredient of the inner layer 204 may be semisolid, gel, or liquid, and the ingredient of the outer layer 206 confines the ingredient of the inner layer 204 against the animal food ingredient 202.

In another example, as is shown in FIG. 3, an animal treatment product 300 can include an animal food ingredient 302 having a cellular energy inhibitor 304 mixed throughout at least a portion of the animal food ingredient 302. The cellular energy inhibitor 304 can be evenly distributed throughout the animal food ingredient 302, evenly distributed throughout a portion of the animal food ingredient, or unevenly distributed throughout, or unevenly distributed throughout a portion of the animal food ingredient 302. In some cases, the cellular energy inhibitor 304 can additionally be absent from one or more portions of the animal food ingredient 302.

In yet another example, as is shown in FIG. 4, an animal treatment product 400 can include an animal food ingredient 402 having a cellular energy inhibitor 404 that includes an outer layer 406, that is either an ingredient of the cellular energy inhibitor 404 or a separate ingredient. In one such configuration, the outer layer 406 can be an ingredient of the cellular energy inhibitor that is reactive with the animal food ingredient 402 and is thus separated therefrom by another ingredient of the cellular energy inhibitor that is nonreactive or less reactive compared to the outer layer ingredient. In another configuration, the ingredients of the cellular energy inhibitor may be reactive with one another, and as such, are maintained separately until broken up by the target animal. In such cases and third layer can also be disposed between the animal food ingredient 404 and the outer layer 406 (not shown). In other configurations, the ingredient of the inner portion 404 may be semisolid, gel, or liquid, and the ingredient of the outer layer 206 confines the ingredient of the inner portion 404 against the animal food ingredient 402.

In a further example, as is shown in FIG. 5, an animal treatment product 500 can include an animal food ingredient 502 surrounding a cellular energy inhibitor 504. In this configuration, the cellular energy inhibitor 504 can contact the animal food ingredient 502 or an intervening layer can be disposed therebetween (not shown). In one example, the cellular energy inhibitor 504 can be solid or substantially solid. In another example, the cellular energy inhibitor 504 can be a semisolid, gel, or liquid, that is contained by the animal food ingredient 502.

The shelf life of the animal food ingredient and/or the 3-BP composition can be enhanced when packaged in an oxygen free environment that is, in some cases, shielded from light. Depending on the various components of the animal treatment product, packaging that substantially precludes moisture can also be beneficial. One example of packaging is Alu-plastic packaging (e.g., Alu-plastic blister pack), which can be used to seal the components of the animal treatment product in an environment that is substantially free of oxygen, where light exposure is not detrimental to the cellular energy inhibitor. Some cellular energy inhibitors, however, can be light sensitive, and as such, a packaging that minimizes light exposure can increase the shelf-life of this component of the animal treatment product. 3-BP is one example of a cellular energy inhibitor that benefits from a light-free environment. Such a light and oxygen-free environment can be provided using Alu-Alu packaging (e.g., Alu-Alu blister pack), where a layer of aluminum foil covers both sides of the package.

Additionally, the animal treatment product can be packaged to be refrigerated, frozen, or maintained at room temperature. In some cases, the temperature at which the animal treatment product is stored can affect shelf-life, which may or may not matter to the owner depending on the intended storage duration prior to use.

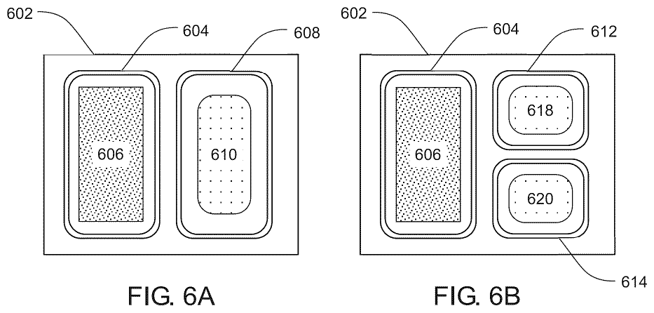

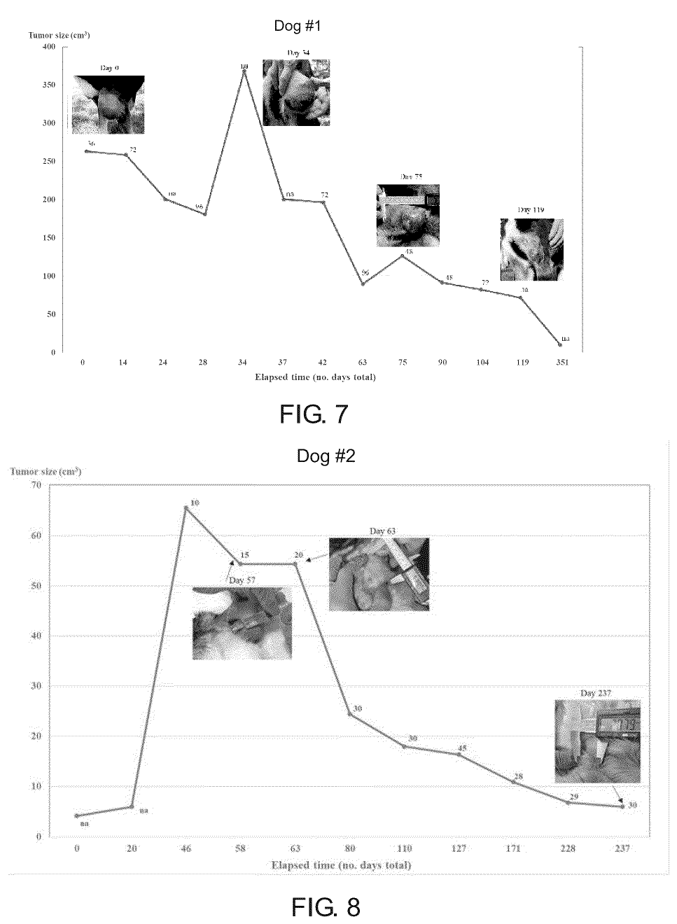

In the examples shown in FIGS. 6A and 6B, packages are shown that are used to contain the animal nutritional product. FIG. 6A, for example, shows a package 602 having a first compartment 604 to contain the animal food ingredient 606. The packaging can be any type of suitable packaging, including Alu-Plastic, Alu-Alu, or the like. The package 602 additionally includes a second compartment 608 containing a cellular energy inhibitor 610 sealed therein. FIG. 6B shows an example of a package 602 having a first compartment 604 to contain the animal food ingredient 606. The packaging can be any type of suitable packaging, including Alu-Plastic, Alu-Alu, or the like. The package 602 additionally includes a second compartment 612 and a third compartment 614, where the second compartment 618 includes 3-BP and one or more ingredients of the cellular energy inhibitor and the third compartment 614 includes one or more ingredients of the cellular energy inhibitor, each separably sealed therein.

### EXAMPLES

### Example 1: Dog #1

A dog having an inoperable spontaneous soft tissue sarcoma was treated with a 3-BP composition by both oral and IV routes. The sarcoma initially had a 263.9 cm³ tumor volume, was highly vascularized and aggressive. Tumor volume had decreased to 69.3 (74%) after 11 intertumoral injections of the 3-BP composition over 4 months.

FIG. 7 shows the progress of dog #1 over the time course of the treatment. The images show tumor size (cm³) along the y-axis measured at 13 observation days on the x-axis. Data point labels represent the 3-BP composition injection volume. The tumor volume was down 97% following the 13^{th} injection.

### Example 2: Dog #2

Dog #2 was treated with a 3-BP composition via oral and intratumoral routes for anal sac adenocarcinoma. This adenocarcinoma was aggressive and doubling every two weeks. The tumor decreased in volume over time as with periodic administration of the 3-BP composition, from 67 cm³ to 4 cm³ at day 237. FIG. 8 shows tumor size (cm³) along the y-axis measured at eleven observation days on the x-axis. Data point labels represent the 3-BP composition injection volume.

### Example 3: Dog #3

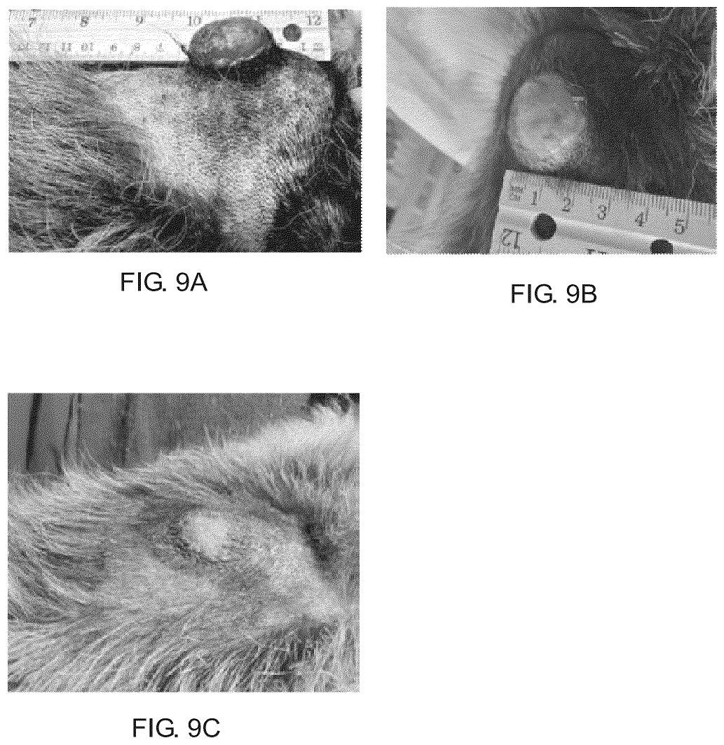

Dog #3 was treated with a 3-BP composition via oral administration for sarcoma. The sarcoma initially had a size of 10×8×5 cm, and was a hard fixed sarcoma on midline of proximal chest. The tumor had ruptured as shown in FIG. 9A. Dog #3 exhibited poor appetite, lethargy and depression. Oral administration of the 3-BP composition began at the time of the initial observation and continued for four months. After the first month, there had been a dramatic improvement with the tumor having been reduced in size to 5×4×3 cm. (see FIG. 9B) The tumor had softened with the ruptured area still healing, as is shown in FIG. 9C. Dog #3's energy and appetite had improved. After 4 months of using oral 3-BP, the tumor was no longer present, and after 8 months only scar tissue and some extra skill remained.

The following examples pertain to specific embodiments and point out specific features, elements, or steps that can be used or otherwise combined in achieving such embodiments.

In one example, an animal treatment product can include a food ingredient and a cellular energy inhibitor combined with the food ingredient. The cellular energy inhibitor can include, for example, a 3-halopyruvate molecule according to Formula I.

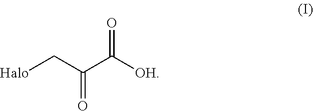

and at least one sugar that stabilizes the 3-halopyruvate molecule by substantially preventing the 3-halopyruvate molecule from hydrolyzing.

In another example, the cellular energy inhibitor can include a 3-bromopyruvate (3-BP) molecule according to formula (II)

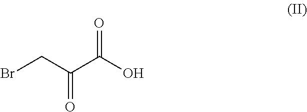

and at least one sugar that stabilizes the 3-BP by substantially preventing the 3-BP from hydrolyzing.

In one example, the animal nutritional product is a dog food formulation. Such a dog food formulation can include the cellular energy inhibitor and a protein. Nonlimiting examples of suitable protein can include meat, appetein, blood serum, blood plasma, albumin, globulin, and the like, including mixtures thereof. Suitable meat protein can include, without limitation, poultry, fish, beef, lamb, duck, or the like, including combinations thereof. A dog food formulation can further include a variety of additional ingredients that can vary depending on a given dog food formulation. Such ingredients can include, without limitation, carbohydrates, fruits, vegetables, grains, fatty acids, and the like, including combinations thereof.

In another example, the animal nutritional product is a cat food formulation. Such a cat food formulation can include the cellular energy inhibitor and a protein, such as a meat protein, flaxseed meal, or the like, including combinations thereof. The meat protein can include any meat protein such as, without limitation, poultry, fish, beef, lamb, duck, or the like, including combinations thereof. A cat food formulation can further include a variety of additional ingredients that can vary depending on a given cat food formulation. Such ingredients can include, without limitation, carbohydrates, fruits, grains, fatty acids, omega-3 fatty acids, eggs, and the like, including combinations thereof.

In another example, the animal nutritional product is a bird food formulation. Such a bird food formulation can include the cellular energy inhibitor and a variety of ingredients, such as, for example, seeds, nuts, cracked corn, millet, dried fruit, and the like, including combinations thereof.

In another example, the animal nutritional product is livestock feed. Such a livestock feed formulation can include the cellular energy inhibitor and a livestock feed ingredient. Livestock feed ingredients can be highly variable depending on the preference of the owner and the nature of the livestock. General examples of livestock feed ingredients can include, without limitation, corn, soybean meal, dried and wet distillers' grains, bakery meal, corn gluten feed, cottonseed meal, wheat midds, grain sorghum, soybean hulls, oats, fruits, animal protein products, marine products, milk product, wheat products, and the like, including combinations thereof.

In another example, the animal nutritional product is a horse feed. Such a horse feed formulation can include the cellular energy inhibitor and a horse feed ingredient. Horse feed ingredients can be highly variable depending on the preference of the owner and the nature of the horse. General examples of horse feed ingredients can include, without limitation, grains such as oats, barley, corn, rice or wheat and the co-products of these grains such as corn distillers grains, rice bran or wheat midds, pasture hay, wheaten and oaten hay and chaff, sunflower meal, cottonseed meal, soyabean meal, sorghum, pollard (wheat product) horse feed, horse pellets, fruits, and the like, including combinations thereof.

In one example, the at least one sugar can be selected from gluconic acid, glucuronic acid, mannitol, erythritol, isomalt, lactitol, maltitol, sorbitol, xylitol, dulcitol, ribitol, inositol, myo inositol, glycerol, ethylene glycol, threitol, arabitol, galactitol, fucitol, iditol, volemitol, maltotriitol, maltotetraitol, polyglycitol, or a combination thereof.

In one example, the at least one sugar can be a five-carbon sugar.

In one example, the at least one sugar can be at least two five-carbon sugars.

In one example, the cellular energy inhibitor can include a second sugar selected from mannitol, erytritol, isomalt, lactitol, maltitol, sorbitol, xyolitol, dulcitol, ribitol, inositol, myo inositol, sorbitol, and combinations thereof.

In one example, the cellular energy inhibitor can include a second sugar and a third sugar independently selected from mannitol, erytritol, isomalt, lactitol, maltitol, sorbitol, xyolitol, dulcitol, ribitol, inositol, myo inositol, sorbitol, and combinations thereof.

In one example, the at least one sugar can include glycerol, myo inositol, and sorbitol.

In one example, the cellular energy inhibitor can include one or more sugars in a range from about 0.5 wt % to about 50.0 wt % or from about 1.0 wt % to about 25.5 wt %. In yet another example, a cellular energy inhibitor can include one or more sugars in a range from about 0.2 wt % to about 75.0 wt % or from about 0.5 wt % to about 50.0 wt %. In a further example, a cellular energy inhibitor can include one or more sugars in a range from about 0.1 wt % to about 25.0 wt %, from about 0.2 wt % to about 10.0 wt %.

In some examples, the cellular energy inhibitor can include glycerol in a range from about 0.1 wt % to about 5.0 wt % or from about 0.1 wt % to about 3.0 wt %. In other examples, the cellular energy inhibitor can include inositol in a range from about 0.1 wt % to about 10 wt %, from about 0.1 wt % to about 6 wt %. In further examples, the cellular energy inhibitor can include sorbitol in a range from about 0.1 wt % to about 40.0 wt % or from about 0.1 wt % to about 30 wt %. In yet further examples, the cellular energy inhibitor can include mannitol in a range from about 0.1 wt % to about 30 wt % or from about 0.1 wt % to about 10 wt %. Additionally, each of the sugars may be added in a volume up to a maximum solubility of the sugar in the formulation or cellular energy inhibitor.

In one example, the cellular energy inhibitor can include d-lactic acid and epinephrine.

In one example, the cellular energy inhibitor can include a biological buffer that is present in an amount sufficient to at least partially deacidify and neutralize metabolic by-products of the 3-BP molecule.

In one example, the biological buffer is selected from one or more of a citrate buffer, a phosphate buffer, and an acetate buffer.

In one example, the biological buffer is a citrate buffer.

In one example, the biological buffer is a phosphate buffer.

In one example, the animal nutritional product is used to treat cancer condition in the animal.

In another example, the animal treatment product is used as an adjuvant to prevent cancer condition in the animal.

In one example, a method for treating an animal for a condition is provided, including delivering to the animal an animal treatment product including a food ingredient and a cellular energy inhibitor combined with the food ingredient, and allowing the animal to ingest the animal treatment product. The cellular energy inhibitor can include, for example, a 3-bromopyruvic acid and/or its salt 3-bromopyruvate (collectively referred to as 3-BP) according to Formula I,

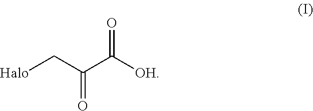

and at least one sugar that stabilizes the 3-BP molecule by substantially preventing the 3-halopyruvate molecule from hydrolyzing.

In one example, the animal nutritional product is a dog food formulation. Such a dog food formulation can include the cellular energy inhibitor and a protein. Nonlimiting examples of suitable protein can include meat, appetein, blood serum, blood plasma, albumin, globulin, and the like, including mixtures thereof. Suitable meat protein can include, without limitation, poultry, fish, beef, lamb, duck, or the like, including combinations thereof. A dog food formulation can further include a variety of additional ingredients that can vary depending on a given dog food formulation. Such ingredients can include, without limitation, carbohydrates, fruits, vegetables, grains, fatty acids, and the like, including combinations thereof.

In another example, the animal nutritional product is a cat food formulation. Such a cat food formulation can include the cellular energy inhibitor and a protein, such as a meat protein, flaxseed meal, or the like, including combinations thereof. The meat protein can include any meat protein such as, without limitation, poultry, fish, beef, lamb, duck, or the like, including combinations thereof. A cat food formulation can further include a variety of additional ingredients that can vary depending on a given cat food formulation. Such ingredients can include, without limitation, carbohydrates, fruits, grains, fatty acids, omega-3 fatty acids, eggs, and the like, including combinations thereof.

In another example, the animal nutritional product is a bird food formulation. Such a bird food formulation can include the cellular energy inhibitor and a variety of ingredients, such as, for example, seeds, nuts, cracked corn, millet, dried fruit, and the like, including combinations thereof.

In another example, the animal nutritional product is livestock feed. Such a livestock feed formulation can include the cellular energy inhibitor and a livestock feed ingredient. Livestock feed ingredients can be highly variable depending on the preference of the owner and the nature of the livestock. General examples of livestock feed ingredients can include, without limitation, corn, soybean meal, dried and wet distillers' grains, bakery meal, corn gluten feed, cottonseed meal, wheat midds, grain sorghum, soybean hulls, oats, fruits, animal protein products, marine products, milk product, wheat products, and the like, including combinations thereof.

In another example, the animal nutritional product is a horse feed. Such a horse feed formulation can include the cellular energy inhibitor and a horse feed ingredient. Horse feed ingredients can be highly variable depending on the preference of the owner and the nature of the horse. General examples of horse feed ingredients can include, without limitation, grains such as oats, barley, corn, rice or wheat and the co-products of these grains such as corn distillers grains, rice bran or wheat midds, pasture hay, wheaten and oaten hay and chaff, sunflower meal, cottonseed meal, soyabean meal, sorghum, pollard (wheat product) horse feed, horse pellets, fruits, and the like, including combinations thereof.

In one example, the at least one sugar can be selected from gluconic acid, glucuronic acid, mannitol, erythritol, isomalt, lactitol, maltitol, sorbitol, xylitol, dulcitol, ribitol, inositol, myo inositol, glycerol, ethylene glycol, threitol, arabitol, galactitol, fucitol, iditol, volemitol, maltotriitol, maltotetraitol, polyglycitol, or a combination thereof.

In one example, the at least one sugar can be a five-carbon sugar.

In one example, the at least one sugar can be at least two five-carbon sugars.

In one example, the cellular energy inhibitor can include a second sugar selected from mannitol, erytritol, isomalt, lactitol, maltitol, sorbitol, xyolitol, dulcitol, ribitol, inositol, myo inositol, sorbitol, and combinations thereof.

In one example, the cellular energy inhibitor can include a second sugar and a third sugar independently selected from mannitol, erytritol, isomalt, lactitol, maltitol, sorbitol, xyolitol, dulcitol, ribitol, inositol, myo inositol, sorbitol, and combinations thereof.

In one example, the at least one sugar can include glycerol, myo inositol, and sorbitol.

In one example, the cellular energy inhibitor can include one or more sugars in a range from about 0.5 wt % to about 50.0 wt % or from about 1.0 wt % to about 25.5 wt %. In yet another example, a cellular energy inhibitor can include one or more sugars in a range from about 0.2 wt % to about 75.0 wt % or from about 0.5 wt % to about 50.0 wt %. In a further example, a cellular energy inhibitor can include one or more sugars in a range from about 0.1 wt % to about 25.0 wt %, from about 0.2 wt % to about 10.0 wt %.

In some examples, the cellular energy inhibitor can include glycerol in a range from about 0.1 wt % to about 5.0 wt % or from about 0.1 wt % to about 3.0 wt %. In other examples, the cellular energy inhibitor can include inositol in a range from about 0.1 wt % to about 10 wt %, from about 0.1 wt % to about 6 wt %. In further examples, the cellular energy inhibitor can include sorbitol in a range from about 0.1 wt % to about 40.0 wt % or from about 0.1 wt % to about 30 wt %. In yet further examples, the cellular energy inhibitor can include mannitol in a range from about 0.1 wt % to about 30 wt % or from about 0.1 wt % to about 10 wt %. Additionally, each of the sugars may be added in a volume up to a maximum solubility of the sugar in the formulation or cellular energy inhibitor.

In one example, the cellular energy inhibitor can include d-lactic acid and epinephrine.

In one example, the cellular energy inhibitor can include a biological buffer that is present in an amount sufficient to at least partially deacidify and neutralize metabolic by-products of the 3-BP molecule.

In one example, the biological buffer is selected from one or more of a citrate buffer, a phosphate buffer, and an acetate buffer.

In one example, the biological buffer is a citrate buffer.

In one example, the biological buffer is a phosphate buffer.

In one example, the animal nutritional product is used to treat cancer condition in the animal.

In another example, the animal treatment product is delivered to the animal as an adjuvant.

In another example, the condition is selected from cancers, autoimmune diseases, pathogenic infections, or a combination thereof.

In yet another example, the condition is selected from canine atopic dermatitis, inflammatory bowel disease, kidney disease, pemphigus foliaceus, atopic dermatitis, lupus, bullous autoimmune skin diseases, immune-mediated polyarthritis and rheumatoid arthritis, or a combination thereof.

In another example, the condition is a cancer selected from sarcomas, canine spindle cell soft sarcomas, bone tumors, anal sac adenocarcinomas, leukemia, lymphoma, myeloma, pancreatic cancer, mast cell tumors, oral tumors, nasal tumors, bladder cancers, hemangiosarcomas, liver cancers, thyroid cancers, stomach cancers, or a combination thereof.

Another example, the animal is a canine.

In another example, the animal is selected from livestock or horses.

What is claimed is:

1. An animal treatment product, comprising:
an animal food product;
a cellular energy inhibitor composition combined with the animal food product, wherein the cellular energy inhibitor composition includes;
3-bromopyruvic acid (3-BP), a 3-BP salt, or a combination thereof;
at least one sugar to stabilize the 3-BP molecule by substantially preventing the 3-BP molecule from hydrolyzing; and
a biological buffer present in an amount sufficient to at least partially deacidify and neutralize metabolic by-products of the 3-BP.

2. The animal treatment product of claim 1, wherein the at least one sugar is a member selected from the group consisting of gluconic acid, glucuronic acid, mannitol, erythritol, isomalt, lactitol, maltitol, sorbitol, xylitol, dulcitol, ribitol, inositol, glycerol, ethylene glycol, threitol, arabitol, galactitol, fucitol, iditol, volemitol, maltotriitol, maltotetraitol, polyglycitol, and combinations thereof.

3. The animal treatment product of claim 1, wherein the cellular energy inhibitor composition further comprises a second sugar selected from the group consisting of mannitol, erythritol, isomalt, lactitol, maltitol, sorbitol, xylitol, dulcitol, ribitol, inositol, sorbitol, and combinations thereof.

4. The animal treatment product of claim 1, wherein the cellular energy inhibitor composition further comprises a second sugar and a third sugar independently selected from mannitol, erytritol, isomalt, lactitol, maltitol, sorbitol, xyolitol, dulcitol, ribitol, inositol, sorbitol, or a combination thereof.

5. The animal treatment product of claim 1, wherein the cellular energy inhibitor composition further comprises at least one sugar selected from glycerol, inositol, and sorbitol.

6. The animal treatment product of claim 1, wherein the cellular energy inhibitor composition further comprises d-lactic acid and epinephrine.

7. The animal treatment product of claim 1, wherein the cellular energy inhibitor composition further comprises a glycolysis inhibitor.

8. The animal treatment product of claim 9, wherein the glycolysis inhibitor is 2-deoxglucose at a concentration from about 1 mM to about 5 mM.

9. The animal treatment product of claim 1, wherein the biological buffer is selected from a citrate buffer, a phosphate buffer, and an acetate buffer.

10. The animal treatment product of claim 1, wherein the biological buffer is a citrate buffer.

11. The animal treatment product of claim 1, wherein the animal food product is selected from meat, carbohydrates, fruits, vegetables, grains, fatty acids, eggs, appetein, blood serum, blood plasma, albumin, globulin, or a combination thereof.

12. The animal treatment product of claim 1, wherein the cellular energy inhibitor composition further comprises at least one additive selected from phospholipids; liposomes; nanoparticles; immune system modulators and/or immune system boosters including brown rice extract, muramyl dipeptide including analogues, mushroom extract, bioflavonoids, Vitamin D3-Binding Protein-Derived Macrophage Activating Factor (GcMAF), inhibitors of nagalase, threonine attached to N-acetylgalactosamine, and antibodies against nagalase; L-lactate dehydrogenase; D-lactate dehydrogenase; nicotinamide adenine dinucleotides; inhibitors for DNA replication; inhibitors for DNA binding; inhibitors for DNA transcription; inhibitors for cell cycle, growth and/or proliferation; inhibitors for signal transduction pathways; inhibitors for angiogensis; small RNAs that interfere with normal gene control including antisense RNA, micro RNA, small hairpin RNA, short hairpin RNA, small interfering RNA; vitamin C; nutritional supplements including vitamins, CoQ10, flavonoids, free fatty acid, alpha lipoic acid, acai, goji, mango, pomegranate, L-carnitine, selenium; a less biologically active amino acid as compared to its isomer; or a combination thereof.

13. The animal treatment product of claim 1, wherein the cellular energy inhibitor composition further comprises a hexokinase inhibitor.

14. A method for treating an animal for a condition, comprising:
providing the animal treatment product of claim 1 to an animal having the condition; and
allowing the animal to ingest the animal treatment product.

15. The method of claim 14, wherein the animal treatment product is provided to the animal as an adjuvant.

16. The method of claim 14, wherein the condition is selected from cancers, autoimmune diseases, pathogenic infections, or a combination thereof.

17. The method of claim 14, wherein the condition is selected from canine atopic dermatitis, inflammatory bowel disease, kidney disease, pemphigus foliaceus, atopic dermatitis, lupus, bullous autoimmune skin diseases, immune-mediated polyarthritis and rheumatoid arthritis, or a combination thereof.

18. The method of claim 14, wherein the condition is a cancer selected from sarcomas, canine spindle cell soft sarcomas, bone tumors, anal sac adenocarcinomas, leukemia, lymphoma, myeloma, pancreatic cancer, mast cell tumors, oral tumors, nasal tumors, bladder cancers, hemangiosarcomas, liver cancers, thyroid cancers, stomach cancers, or a combination thereof.

19. The method of claim 14, wherein the animal is a canine.

20. The method of claim 14, wherein the animal is selected from livestock or horses.


SEQUENCE LISTING (10 sequences)

SEQ ID NO: 1 - protein, 917 aa, Homo sapiens
MIAAQLLAYYFTELKDDQVKKIDKYLYAMRLSDETLIDIMTRFRKEMKNGLSRDFNPTATVKMLPTFVRSIPDGSEKGDFIALDLGGSSFRILRVQVNHEKNQNVHMESEVYDTPENIVHGSGSQLFDHVAECLGDFMEKRKIKDKKLPVGFTFSFPCQQSKIDEAILITWTKRFKASGVEGADVVKLLNKAIKKRGDYDANIVAVVNDTVGTMMTCGYDDQHCEVGLIIGTGTNACYMEELRHIDLVEGDEGRMCINTEWGAFGDDGSLEDIRTEFDREIDRGSLNPGKQLFEKMVSGMYLGELVRLILVKMAKEGLLFEGRITPELLTRGKFNTSDVSAIEKNKEGLHNAKEILTRLGVEPSDDDCVSVQHVCTIVSFRSANLVAATLGAILNRLRDNKGTPRLRTTVGVDGSLYKTHPQYSRRFHKTLRRLVPDSDVRFLLSESGSGKGAAMVTAVAYRLAEQHRQIEETLAHFHLTKDMLLEVKKRMRAEMELGLRKQTHNNAVVKMLPSFVRRTPDGTENGDFLALDLGGTNFRVLLVKIRSGKKRTVEMHNKIYAIPIEIMQGTGEELFDHIVSCISDFLDYMGIKGPRMPLGFTFSFPCQQTSLDAGILITWTKGFKATDCVGHDVVTLLRDAIKRREEFDLDVVAVVNDTVGTMMTCAYEEPTCEVGLIVGTGSNACYMEEMKNVEMVEGDQGQMCINMEWGAFGDNGCLDDIRTHYDRLVDEYSLNAGKQRYEKMISGMYLGEIVRNILIDFTKKGFLFRGQISETLKTRGIFETKFLSQIESDRLALLQVRAILQQLGLNSTCDDSILVKTVCGVVSRRAAQLCGAGMAAVVDKIRENRGLDRLNVTVGVDGTLYKLHPHFSRIMHQTVKELSPKCNVSFLLSEDGSGKGAALITAVGVRLRTEASS

SEQ ID NO: 2 - protein, 916 aa, Homo sapiens
MDCEHSLSLPCRGAEAWEIGIDKYLYAMRLSDETLIDIMTRFRKEMKNGLSRDFNPTATVKMLPTFVRSIPDGSEKGDFIALDLGGSSFRILRVQVNHEKNQNVHMESEVYDTPENIVHGSGSQLFDHVAECLGDFMEKRKIKDKKLPVGFTFSFPCQQSKIDEAILITWTKRFKASGVEGADVVKLLNKAIKKRGDYDANIVAVVNDTVGTMMTCGYDDQHCEVGLIIGTGTNACYMEELRHIDLVEGDEGRMCINTEWGAFGDDGSLEDIRTEFDREIDRGSLNPGKQLFEKMVSGMYLGELVRLILVKMAKEGLLFEGRITPELLTRGKFNTSDVSAIEKNKEGLHNAKEILTRLGVEPSDDDCVSVQHVCTIVSFRSANLVAATLGAILNRLRDNKGTPRLRTTVGVDGSLYKTHPQYSRRFHKTLRRLVPDSDVRFLLSESGSGKGAAMVTAVAYRLAEQHRQIEETLAHFHLTKDMLLEVKKRMRAEMELGLRKQTHNNAVVKMLPSFVRRTPDGTENGDFLALDLGGTNFRVLLVKIRSGKKRTVEMHNKIYAIPIEIMQGTGEELFDHIVSCISDFLDYMGIKGPRMPLGFTFSFPCQQTSLDAGILITWTKGFKATDCVGHDVVTLLRDAIKRREEFDLDVVAVVNDTVGTMMTCAYEEPTCEVGLIVGTGSNACYMEEMKNVEMVEGDQGQMCINMEWGAFGDNGCLDDIRTHYDRLVDEYSLNAGKQRYEKMISGMYLGEIVRNILIDFTKKGFLFRGQISETLKTRGIFETKFLSQIESDRLALLQVRAILQQLGLNSTCDDSILVKTVCGVVSRRAAQLCGAGMAAVVDKIRENRGLDRLNVTVGVDGTLYKLHPHFSRIMHQTVKELSPKCNVSFLLSEDGSGKGAALITAVGVRLRTEASS

SEQ ID NO: 3 - protein, 921 aa, Homo sapiens
MGQICQRESATAAEKPKLHLLAESEIDKYLYAMRLSDETLIDIMTRFRKEMKNGLSRDFNPTATVKMLPTFVRSIPDGSEKGDFIALDLGGSSFRILRVQVNHEKNQNVHMESEVYDTPENIVHGSGSQLFDHVAECLGDFMEKRKIKDKKLPVGFTFSFPCQQSKIDEAILITWTKRFKASGVEGADVVKLLNKAIKKRGDYDANIVAVVNDTVGTMMTCGYDDQHCEVGLIIGTGTNACYMEELRHIDLVEGDEGRMCINTEWGAFGDDGSLEDIRTEFDREIDRGSLNPGKQLFEKMVSGMYLGELVRLILVKMAKEGLLFEGRITPELLTRGKFNTSDVSAIEKNKEGLHNAKEILTRLGVEPSDDDCVSVQHVCTIVSFRSANLVAATLGAILNRLRDNKGTPRLRTTVGVDGSLYKTHPQYSRRFHKTLRRLVPDSDVRFLLSESGSGKGAAMVTAVAYRLAEQHRQIEETLAHFHLTKDMLLEVKKRMRAEMELGLRKQTHNNAVVKMLPSFVRRTPDGTENGDFLALDLGGTNFRVLLVKIRSGKKRTVEMHNKIYAIPIEIMQGTGEELFDHIVSCISDFLDYMGIKGPRMPLGFTFSFPCQQTSLDAGILITWTKGFKATDCVGHDVVTLLRDAIKRREEFDLDVVAVVNDTVGTMMTCAYEEPTCEVGLIVGTGSNACYMEEMKNVEMVEGDQGQMCINMEWGAFGDNGCLDDIRTHYDRLVDEYSLNAGKQRYEKMISGMYLGEIVRNILIDFTKKGFLFRGQISETLKTRGIFETKFLSQIESDRLALLQVRAILQQLGLNSTCDDSILVKTVCGVVSRRAAQLCGAGMAAVVDKIRENRGLDRLNVTVGVDGTLYKLHPHFSRIMHQTVKELSPKCNVSFLLSEDGSGKGAALITAVGVRLRTEASS

SEQ ID NO: 4 - protein, 905 aa, Homo sapiens
MAKRALRDFIDKYLYAMRLSDETLIDIMTRFRKEMKNGLSRDFNPTATVKMLPTFVRSIPDGSEKGDFIALDLGGSSFRILRVQVNHEKNQNVHMESEVYDTPENIVHGSGSQLFDHVAECLGDFMEKRKIKDKKLPVGFTFSFPCQQSKIDEAILITWTKRFKASGVEGADVVKLLNKAIKKRGDYDANIVAVVNDTVGTMMTCGYDDQHCEVGLIIGTGTNACYMEELRHIDLVEGDEGRMCINTEWGAFGDDGSLEDIRTEFDREIDRGSLNPGKQLFEKMVSGMYLGELVRLILVKMAKEGLLFEGRITPELLTRGKFNTSDVSAIEKNKEGLHNAKEILTRLGVEPSDDDCVSVQHVCTIVSFRSANLVAATLGAILNRLRDNKGTPRLRTTVGVDGSLYKTHPQYSRRFHKTLRRLVPDSDVRFLLSESGSGKGAAMVTAVAYRLAEQHRQIEETLAHFHLTKDMLLEVKKRMRAEMELGLRKQTHNNAVVKMLPSFVRRTPDGTENGDFLALDLGGTNFRVLLVKIRSGKKRTVEMHNKIYAIPIEIMQGTGEELFDHIVSCISDFLDYMGIKGPRMPLGFTFSFPCQQTSLDAGILITWTKGFKATDCVGHDVVTLLRDAIKRREEFDLDVVAVVNDTVGTMMTCAYEEPTCEVGLIVGTGSNACYMEEMKNVEMVEGDQGQMCINMEWGAFGDNGCLDDIRTHYDRLVDEYSLNAGKQRYEKMISGMYLGEIVRNILIDFTKKGFLFRGQISETLKTRGIFETKFLSQIESDRLALLQVRAILQQLGLNSTCDDSILVKTVCGVVSRRAAQLCGAGMAAVVDKIRENRGLDRLNVTVGVDGTLYKLHPHFSRIMHQTVKELSPKCNVSFLLSEDGSGKGAALITAVGVRLRTEASS

SEQ ID NO: 5 - protein, 917 aa, Homo sapiens
MIASHLLAYFFTELNHDQVQKVDQYLYHMRLSDETLLEISKRFRKEMEKGLGATTHPTAAVKMLPTFVRSTPDGTEHGEFLALDLGGTNFRVLWVKVTDNGLQKVEMENQIYAIPEDIMRGSGTQLFDHIAECLANFMDKLQIKDKKLPLGFTFSFPCHQTKLDESFLVSWTKGFKSSGVEGRDVVALIRKAIQRRGDFDIDIVAVVNDTVGTMMTCGYDDHNCEIGLIVGTGSNACYMEEMRHIDMVEGDEGRMCINMEWGAFGDDGSLNDIRTEFDQEIDMGSLNPGKQLFEKMISGMYMGELVRLILVKMAKEELLFGGKLSPELLNTGRFETKDISDIEGEKDGIRKAREVLMRLGLDPTQEDCVATHRICQIVSTRSASLCAATLAAVLQRIKENKGEERLRSTIGVDGSVYKKHPHFAKRLHKTVRRLVPGCDVRFLRSEDGSGKGAAMVTAVAYRLADQHRARQKTLEHLQLSHDQLLEVKRRMKVEMERGLSKETHASAPVKMLPTYVCATPDGTEKGDFLALDLGGTNFRVLLVRVRNGKWGGVEMHNKIYAIPQEVMHGTGDELFDHIVQCIADFLEYMGMKGVSLPLGFTFSFPCQQNSLDESILLKWTKGFKASGCEGEDVVTLLKEAIHRREEFDLDVVAVVNDTVGTMMTCGFEDPHCEVGLIVGTGSNACYMEEMRNVELVEGEEGRMCVNMEWGAFGDNGCLDDFRTEFDVAVDELSLNPGKQRFEKMISGMYLGEIVRNILIDFTKRGLLFRGRISERLKTRGIFETKFLSQIESDCLALLQVRAILQHLGLESTCDDSIIVKEVCTVVARRAAQLCGAGMAAVVDRIRENRGLDALKVTVGVDGTLYKLHPHFAKVMHETVKDLAPKCDVSFLQSEDGSGKGAALITAVACRIREAGQR

SEQ ID NO: 6 - protein, 25 aa, Homo sapiens
MIAAQLLAYYFTELKDDQVKKIDKY

SEQ ID NO: 7 - protein, 25 aa, Homo sapiens
MDCEHSLSLPCRGAEAWEIGIDKYL

SEQ ID NO: 8 - protein, 25 aa, Homo sapiens
MGQICQRESATAAEKPKLHLLAESE

SEQ ID NO: 9 - protein, 25 aa, Homo sapiens
MAKRALRDFIDKYLYAMRLSDETLI

SEQ ID NO: 10 - protein, 25 aa, Homo sapiens
MIASHLLAYFFTELNHDQVQKVDQY